(19) United States
(12) Patent Application Publication
(10) Pub. No.: US 2020/0323691 A1
(43) Pub. Date: Oct. 15, 2020

(54) DEVICE FOR MAKING AVAILABLE A SKIN OR WOUND DRESSING

(71) Applicant: Derma Tools Biotech GmbH, Roedermark (DE)

(72) Inventors: Michael Wiehl, Erlangen (DE); Peter Keitel, Buch Am Wald (DE); Dirk Kaiser, Eppertshausen (DE); Mark-Andre Freyberg, Darmstadt (DE); Stephan Bruckmann, Dortmund (DE)

(21) Appl. No.: 16/759,015

(22) PCT Filed: Oct. 25, 2018
(86) PCT No.: PCT/EP2018/079299

§ 371 Date: Apr. 24, 2020

(30) Foreign Application Priority Data
Oct. 27, 2017 (DE) 10 2017 125 281.9

(51) Int. Cl. A61F 13/00; A61F 15/00

(52) U.S. Cl. CPC A61F 13/00063; A61F 13/00072; A61F 15/002

(57) ABSTRACT

A device for providing a skin or wound dressing, a wound treatment set, and a method to provide a skin or wound dressing, wherein the skin or wound dressing includes an active substance, and wherein the device comprises at least two chambers for the storage of at least two liquids, which are openable by mechanical action, so that, after opening of the chambers, the liquids are miscible and are transferrable to an active substance carrier.

The present invention relates to a device for providing a skin or wound dressing, a wound treatment set and a method for providing a skin or wound dressing.

Skin or wound dressings which can serve to administer medication have been known for a long time. For example, plaster patch-type systems for medication are described in the documents DE 19733981 A1, EP 2793869 B1, EP 1928436 B1, DE 19503336 A1, DE 19712699 A1 and U.S. Pat. No. 8,784,874. The application forms described therein, frequently relate to medicines which can be easily and safely stored, whereby a mixture of the substances is not necessary in order to achieve a certain effect. The embodiment in U.S. Pat. No. 8,784,874 describes a system where the same or different medications are applied on the surface of various areas, which are then mixed before use by folding. For the presentation of liquid medications which are contained on a skin or wound dressing, the embodiments described therein are not generally suited. This is particularly the case when the active substance itself only has a limited shelf life, however its stability increases after two components have been mixed. Furthermore, the shelf life of various medications can be considerably increased by variations of the pH-value. For the application of the medication, however, there must be certainty that a tolerable pH-value has been adjusted.

Wound plasters which can be removed from the packaging simply and reliably are, among others, disclosed in WO 01/02270 A1 and WO 2014/070589A1. Here, though, only standard plasters as wound dressings are described, whereby they must be stored in a special packaging which can be simply removed. Furthermore, multi-chamber bags are known from the current state of the art, which serve to store various liquids, whose mixtures can be used for example in dialysis treatment, as infusion solutions or for parenteral nutrition. These bags are described, among others, in EP 2167011 B1 and WO 95/26177 A1. However such multi-chamber bags are used for the mixtures of large volumes of liquids, whereby these bags are generally used in the course of inpatient or at least outpatient treatment.

Considering the state of the art, it is therefore object of the present invention to provide a device for providing a skin or wound dressing, via which a liquid active substance, with a limited shelf life, can be safely and reliably administered. Also, the device should ideally be simple to manufacture at low cost.

In particular, the device should be able to be mass produced, and also made available in the form of a disposable item.

Moreover, the provision of a generic device which can be handled easily and safely is a purpose of the present invention. In particular, the device should not only be suitable for use by professional care givers, but also by untrained persons, for example patients, so that they can take care of their own wounds. In this regard, any hazard for the patient should be excluded. Furthermore, the force required and the demands on motor skills when operating the device should be kept relatively low. Here, the handling should be as intuitive as possible so that incorrect handling is excluded as far as possible.

Additionally, the device for provision of a skin or wound dressing should be easily adaptable to dosing requirements, so that, for example, relatively large dosages can be administered. Furthermore, it was also a purpose of the present invention to make a class-specific device available which can also be used for the application of relatively low volumes of liquid. What's more, the device should provide a skin or wound dressing with a predefined active substance dosage, so that it contains a preferably constant active substance concentration. Preferably, the dosing must be carried out so that no liquid active substance can drip out of the wound dressing, or there are areas of skin or wound dressing that are not covered with active substance upon application. For example, the device should provide a skin or wound dressing where a total of 2 ml of liquid are distributed over a surface of ca. 10 cm².

Furthermore, the skin or wound dressing to be provided should not be subject to any particular restriction regarding its size. It should be possible to provide relatively large and relatively small dressings.

Moreover, the device, and the resulting skin or wound dressing, should have a low microbial count, whereby both should be as sterile as possible. Furthermore, the device should show a high rate of acceptance, for example the device should have a high perceived value and the skin or wound dressing should be able to be designed in the color scheme usual in the present field, namely white.

These as well as further objects which are not explicitly mentioned, but which can be easily deduced or derived from the contexts introductorily discussed are solved by a device for providing a skin or wound dressing, containing an active substance, having all the features of patent claim 1.

The subject matter of the present invention is accordingly a device for providing a skin or wound dressing, including an active substance, which is characterized by the device containing at least two chambers for storing at least two liquids, which can be opened by mechanical action, so that after opening the chambers, the liquids are miscible and can be transferred to an active substance carrier.

In particular, the present invention permits the safe and reliable provision of an active substance-containing skin or wound dressing, whose applicable active substance cannot be easily stored over a long period, but which can be preserved by mixing two components which are, as such, stable.

In particular, a device, according to the invention, for providing a skin or wound dressing enables the safe and reliable application of a liquid active substance with a limited shelf life. Here, a device is preferably relative simple and inexpensive to manufacture. Furthermore, a device, according to the invention, can be mass produced. Preferably, a device, according to the invention, can be provided in the form of a disposable item.

Moreover, the device for providing a skin or wound dressing can be handled simply and safely. In particular, the device can not only be used by professional care givers, but also by untrained persons, for example patients, so that a self-treatment of wounds is enabled. In particular, the handling of the preferred embodiments of the present device is so arranged that, when used according to instructions, a hazard for patients is excluded. Furthermore, the force required and the demands on motor skills when handling the device can generally be kept very low. Here, the handling of the preferred embodiments is very intuitive, so that incorrect use can be almost excluded. This ensures proper use, so that an unwanted release of the components stored in the device can only occur in the event of intent or deliberate action.

Furthermore, the device for the provision of a skin or wound dressing can be easily adapted to fulfill dosing requirements, so that relatively high or low dosages can be applied, according to the requirements of the patient. Furthermore, the present invention makes a class-specific device available which can also be used with low volumes of liquid. Furthermore the device provides a skin and wound dressing with a specified dosage, so that the skin or wound dressing preferably demonstrates an almost constant active substance concentration. Preferably, the dosage is adjusted so that no liquid active substance drips out, or leaves areas of the skin or wound dressing without active substance cover. Furthermore, due to the design of the device, it is preferably ensured that only completely mixed liquid volumes come into contact with the wound dressing.

Furthermore, the skin or wound dressing to be provided is not subject to any particular restriction regarding its size. This means that relatively large and relatively small dressings can be provided.

Moreover, the device and the skin or wound dressing provided through it, have a low microbial count, whereby both can be kept sterile. Furthermore, the device can be designed in such a way that a high level of acceptance is achieved among users and, in particular, that it meets the usual standards. The device can thus be designed to give a high perceived value and the skin or wound dressing can be designed in the color scheme customary in the present field, namely white.

The device of the present invention contains at least two chambers for the storage of at least two liquids. Here, the device can have more than two chambers, whereby in these chambers exactly two different liquids are stored, or more than two liquids. Preferably, the number of individual chambers corresponds with the number of different liquids.

After opening of the chambers, the liquids are miscible and transferrable to an active substance carrier. Accordingly, after opening, the chambers are in flow connection with the active substance carrier.

In a special configuration it may be provided that after opening the chambers the liquids are mergeable outside the active substance carrier, preferably by creating by a mixing area upon opening the two chambers, or a mixing area is present, into which the liquids are transferrable and the liquids are subsequently transferrable from the mixing area into the active substance carrier. Preferably, the opening of the two chambers takes place in a first step, and in a separated second step, the mixture obtained is transferred into the active substance carrier by establishing a flow connection between the mixing area and the space in which the active substance carrier is provided.

The minimum of two chambers for the storage of two liquids are openable mechanically. Here, the type of mechanical action is not important, so that this can be carried out with a turning movement or by a linear movement. Therefore, a pulling device or a push-in component, e.g. a knob or the like, may be provided to achieve the movements mentioned.

In particular, it may be provided that the turning or linear movement is provided with a force conversion so that a higher force is applied to the chambers over a longer ovement path. For example, a thread can be provided to effect a linear movement to open the chambers.

In particular, the device can be designed in such a way as to ensure simple and safe operation. Preferably, only one or two control elements are provided. The actuation of these the device mixes the components and transfers them to the active substance carrier. In a preferred embodiment, first of all the chambers with the two liquids are connected by a first opening step and in a subsequent opening step the space in which the mixed liquids are present is connected to the space in which the active substance carrier is provided. These two opening steps can be achieved by actuating one control element or by actuating two control elements. When two control elements are actuated, these separately present elements are preferably arranged in such a way that the control element by which the chambers with two liquids are connected to each other or brought into flow connection must be operated first. This can be achieved, for example, by ensuring that the second control element can only be operated after the first control element has been triggered. Preferably, the second control element is covered by the first control element.

In using such a design, in particular, a simple and safe operation of the device can be achieved, so that untrained persons, or persons with impaired motor skills, can obtain a skin or wound dressing with an active substance, safely and reliably.

Preferably, the two chambers for storing two liquids are openable by pressure, whereby the released liquids are transferrable to the active substance carrier, preferably with the aid of this pressure. This pressure can be generated by direct pressure on a structural element, which is preferably flexible. In a preferable embodiment, the pressure can be achieved by force conversion, for example using a thread in a lid, whereby the turning of the lid reduces the volume below it. The pressure created by the volume reduction opens the chambers.

In a further preferred embodiment, an elastic element can be used which puts part of the device under tension. The energy contained in the tension can be released by loosening a safety holder, which in turn, opens the chambers. Elastic elements are widely known in professional circles, and these elements are preferably used in the form of rubber-containing plastic strips or in the form of elastomers, which may be in the form of films.

Preferably the at least two separate chambers for storing two liquids are openable by a turning movement or linear movement, whereby the chambers are opened mechanically by a protruding edge, a sealed area or by a predetermined breaking point, for example an openable weld seam. For example, a chamber wall can be separated using a protruding edge, preferably the protruding edge is equipped with a sharp edge or point, to facilitate opening this wall. Also, a sealed off area between the chambers can be made mechanically permeable by lifting it. A sealed off area makes up part of the device where the chambers are formed, whereby the parts are so designed to prevent an unintentional substance transfer. Here, the parts of the device which form the chamber are manufactured from corresponding materials, or comprise these materials. Furthermore, one or more seals can be used, which is or are provided between the chambers. Furthermore, the parts of the device forming the chambers can have an appropriate shape, for example comprising sealing lips or similar. In a further embodiment, the chamber preferably has a predetermined breaking point which, depending on the type of chamber material, can be designed, for example, as a thin area or as an openable welding seam (peeling strip).

In addition, it may be provided that a safeguarding mechanism must be released to effect the turning movement or the linear movement. This safeguarding mechanism is preferably provided by a predetermined breaking point, a weld or a seal, the integrity of which indicates the proper condition of the device.

The device according to the present invention includes at least one active substance carrier. Accordingly, the device preferably comprises at least one chamber or a space where the active substance carrier is provided. Here, the space or the chamber is preferably so designed that the active substance carrier can be moistened with the liquids, preferably the mixture thereof, without the liquid leaking from the device. Accordingly, in order to remove the active substance carrier comprising active substance, or rather the skin or wound dressing itself, an additional operation step is necessary. Furthermore, it is possible that the device contains a chamber or casing which encloses the active substance carrier.

In a special embodiment, it is possible that the device has an outer packaging.

In a further embodiment, the liquids are preferably introduceable over the surface of the upper side of the active substance carrier. The upper side has the largest surface area of the three-dimensional active substance carrier.

Furthermore, it may be provided that the two chambers and the active substance carrier are arranged on at least two different levels. In a special embodiment, the two chambers are arranged on one level and the active substance carrier is situated below this level.

In a specific embodiment of the present device, it includes preferably at least one lid which, with an intermediate piece, which encloses the two separate chambers for storing two liquids, or which forms these chambers in the lid or the intermediate piece. In a special embodiment, there are at least two separate chambers for storing two liquids, preferably arranged in a ring, whereby one liquid is in an inner area and the second liquid is situated in the outer area.

Furthermore, it may be provided that the intermediate piece has several openings, preferably is of sieve-like design. Here, the intermediate piece may comprise an impervious inner area or be designed in a sieve-like manner over the entire inner surface.

Also it can be provided that the intermediate piece has a least one, preferably two protrusions, which are formed in such a way that the at least two separate chambers for the storage of two liquids, by moving them in the direction of the protrusions, are openable, whereby the movement in the direction of the protrusions can be a turning movement or a linear movement.

In a further special embodiment of the present device, it can be provided that between the intermediate piece and the lid, a sealed area is formed, whereby for example at least one seal is provided, the parts of the lid and the intermediate piece are made of appropriate materials, or exhibit a sealing shape, such as sealing lips, whereby the parts are mechanically moveable. Preferably two sections, preferably annularly arranged walls, are provided in the lid, which are designed in such a way that, together with other sections of the lid and the intermediate piece, at least two separate chambers are formed for storing two liquids. Here, preferably between the sections of the chambers, which are formed by the intermediate piece, and the sections which are formed by the lid, in particular the walls, arranged in a ring form, at least one seal is provided which is openable by movement of the lid, whereby the opening of the seal can be achieved either by a turning movement or a linear movement, preferably by a turning movement, whereby the lid is lifted in relation to the intermediate piece. Here the seal can also be formed by an appropriate material choice and/or an appropriate shape.

Preferably, the active substance carrier is located below the intermediate piece, whereby the active substance carrier is held by an openable bottom piece. Preferably, the bottom piece is impervious to liquids. Moreover, the bottom piece provides a grip portion which is not covered by the active substance carrier.

In a further embodiment, it can be provided that the device has a casing in which the two chambers are situated, whereby a pull strap is connected to the two chambers and via which, the two chambers can be pulled out of the casing.

In yet another embodiment, it may be provided that the device has a casing in which the two chambers and the active substance carrier are inserted, whereby a lid is provided which is connected to the casing via a turning axis, wherein by opening the lid a pressure is creatable in the chambers by which the chambers are openable.

Furthermore, it may be provided that the two chambers for storage of two liquids and the active substance carrier are arranged on three different levels. Preferably, the active substance carrier is located between the two chambers for storing two liquids. In a further embodiment, one of the chambers is located between a further chamber for storing liquid and the active substance carrier.

In a preferable embodiment, it may be provided that the device includes at least one elastic element, whereby through release of a safety holder, a movement is initiated, preferably a flap mechanism, which facilitates, preferably effects, the opening of the chambers for storing two liquids. In this embodiment, part of the device is under tension, whereby this tension is caused by the elastic element. An embodiment of the present device with at least one elastic element includes preferably at least two plastic layers, which differ with regard to their impact strength, stiffness and/or hardness.

A further embodiment of the present device with at least one elastic element is preferably characterized in that the two chambers for storing two liquids are formed from at least two plastic layers which are firmly connected on two opposite sides and one side, via which the chambers are connectable, is separated by a break-off edge.

Furthermore, in an embodiment of the present device with at least one elastic element it may be provided that the active substance carrier is contained in a third chamber, which is formed by at least two plastic layers which are firmly connected on two opposite sides, and on one side is separated by a break-off edge from at least one further chamber which chamber is formable as a mixing chamber.

In a further embodiment, it may be provided that the device includes three plastic layers, which differ in their mechanical properties, whereby the device preferably has at least one flexible plastic layer A, at least one elastomer containing layer B, and a stiff plastic layer C.

Preferably, the flexible plastic layer A and the elastomer containing layer B are firmly connected together, for example, these layers A and B are preferably two-dimensional welded or glued together or co-extruded.

Furthermore, it may be provided that the flexible plastic layer A and the stiff plastic layer C have a similar area, and that the elastomer containing layer B has a smaller area than the flexible plastic layer A or the stiff plastic layer C. An area is generally similar when the relationship of the areas lies in the range of 1.5:1 to 1:1.5, preferably in the range of 1.2:1 to 1:1.2 and especially preferably in the range of 1.1:1 to 1:1.1, and the length and breadth relationships are also in the range of 1.5:1 to 1:1.5, preferably in the range of 1.2:1 to 1:1.2 and especially preferably in the range of 1.1:1 to 1:1.1. In the case of a flat object, the length represents the largest dimension over the largest surface of an object, the present case of the even layer, the width is the second largest expansion over the largest surface of the flat layer and the thickness, the smallest expansion, for determining the volume of an object.

Furthermore, it may be provided that the flexible plastic layer A and the stiff plastic layer C are connected at the edges, for example welded or glued, so that a cavity is formed between the flexible plastic layer A and the stiff plastic layer C. Preferably, chambers are formed in the cavity which are detachably separated by a kink edge, whereby preferably in one chamber the first liquid is provided and in a second chamber the second liquid is provided, and then in a third chamber, the active substance carrier is provided. In particular, it may be provided that the chamber in which the first liquid is placed, and the chamber in which the second liquid is placed, are separated by a kink edge, so that the chamber with the active substance carrier is only adjacent to one chamber which contains one liquid.

In this embodiment, the elastomer containing layer B in combination with the flexible plastic layer A and the stiff plastic layer C seal off the chambers from each other, preferably by pressure which is particularly present at the kink edge.

With special embodiments of the previously described device with at least one elastic element, after opening the chambers with the two liquids and the chamber with the active substance carrier, pressure can cause the mixed liquid to be actively transferred to the active substance carrier. This pressure is based preferably on the restoring forces which can be provided through the combination of a flexible plastic layer and a stiff plastic layer. In a preferred embodiment, after the opening of the chambers, a planar arrangement of the components of the device is formed. The tension created during the manufacture of the structure due to the strength of the stiff plastic layer is preferably released so that the original flat shape is restored and the liquid is pressed into the active substance carrier. This effect, which can also be called memory effect, is based on the molecular properties of the polymers as well as the manufacturing and/or processing methods of the plastic layers.

In a further embodiment, it may be provided that the two chambers and the active substance carrier are arranged on one level. Preferably, in this embodiment, the device is enclosed in a casing, which contains the two chambers and the active substance carrier, whereby a pull strap is connected with the two chambers, via which the two chambers can be removed from the casing.

The present device for the provision of a skin or wound dressing contains at least two chambers for storing two liquids. These chambers can be very similar or also have different shapes, whereby preferred shapes of the chambers for storing liquids are previously described and are again subsequently described in the drawings, so that this is generally referred to. It can therefore be provided that at least two separate chambers are made from one plastic material, whereby these have preferably at least one part made of a flexible plastic, especially preferred manufactured from a plastic film.

In a special embodiment, the plastic from which the at least two separate chambers, or parts of these chambers, are manufactured is hydrophobic. A hydrophobic plastic exhibits preferably a surface energy of maximum 90 mN/m, specially preferred maximum 65 mN/m and very specially preferred, a maximum of 50 mN/m. Preferably, it may be provided that the plastic exhibits a surface energy in the range of 10 to 90 mN/m, specially preferred in the range of 14 to 65 mN/m and very specially preferred in the range of 18 to 50 mN/m.

As well as the chambers for storing at least two liquids, specially preferred are all parts of the device made of hydrophobic plastic which come into contact with the two liquids or their mixture, whereby the previously detailed surface energies for the chamber materials also apply to these plastics. The nature of the plastic parts which come into contact with the two liquids or their mixture depends on the actual design of the device, whereby the intermediate layer or the bottom piece belong here, as previously and subsequently described.

The surface energy is determined according to the method of Ownes-Wendt-Rabel & Kaelble. For this, the measurement series are carried out with the standard series according to Busscher, whereby the test fluids used are water [SFT 72.1 mN/m], formamide [SFT 56.9 mN/m, diiodomethane [SFT 50.0 mN/m] and alpha-bromonaphthalene [SFT 44.4 mN/m]. The measurement is carried out at 20° C. The surface energy can be determined with a contact angle measuring system G40 from the Krüss company, Hamburg, whereby the performance is described in the user manual of the contact angle measuring system G40, 1993. With regard to calculation methods, reference is made to A. W. Neumann, Über die Messmethodik zur Bestimmung grenzflächenenergetischer Größen (concerning measurement methods for the determination of interfacial energetic values), Part I, Zeitschrift für Phys. Chem. (Journal of Phys. Chem.), Bd. 41, P. 339-352 (1964), and A. W. Neumann, Über die Messmethodik zur Bestimmung grenzflächenenergetischer Größen, Part II, Zeitschrift für Phys. Chem., Bd. 43, P. 71-83 (1964).

Furthermore, it may be provided that the two chambers each have a volume in the range of 0.1 to 10 ml, preferably 0.3 to 5 ml and specially preferred in the range of 0.5 to 2 ml.

Also, it may be provided that the two liquids in the chambers are sterile.

As well as the at least two chambers for storing two liquids, the present device exhibits at least one active substance carrier. The active substance carrier is provided for it to be moistened with a liquid mixture. Accordingly, the active substance carrier is preferably a fabric, particularly preferably a woven fabric, knitted fabric, crotcheted fabric, braided fabric, stitch-bonded fabric, non-woven or felt, particularly preferably a non-woven. The material out of which the active substance carrier is made is not a critical factor, therefore this can be chosen according to requirements. Therefore, the materials which are commonly used in the medical field can be applied, and these will be selected as most appropriate for the liquid with which the active substance carrier is moistened. Here, the liquid preferably demonstrates good compatibility with the material. In this way, an even and adequate dosage of the active substance on the skin or the wound can be ensured. In case the liquid for example has a high polarity, as in particular water, the active substance carrier is preferably hydrophilic.

Among the preferred materials out of which the active substance carrier can be manufactured are, in particular, polyacrylates and polysaccharides, for example cellulose or cellulose derivatives, whereby particularly preferred cellulose or cellulose derivatives, such as for example viscose, are used, whose hydrophily is adapted to the requirements by chemical modification.

A hydrophilic active substance carrier preferably exhibits a surface energy of at least 55 mN/m, particularly preferred at least 63 mN/m and very particularly preferred at least 70 mN/m. Preference may be given to a hydrophilic active substance carrier which has a surface energy in the range of 55 to 150 mN/m, particularly preferred in the range of 63 to 120 mN/m, and very particularly preferred in the range of 70 to 90 mN/m.

The previously defined configurations with regard to the hydrophobicity of the plastics with which the liquids come into contact, such as, for example, the materials out of which the chambers are made, and, where applicable, the material used for the intermediate piece or the bottom piece, as well as the hydrophily of the material from which the active substance carrier was obtained, apply in particular to hydrophilic liquids, preferably for aqueous media, in which the active substance is, or active substances are dissolved or dispersed.

For hydrophobic liquids, for example oils containing active substance, the materials are selected preferably inverse with regard to polarity, so that the plastics which come into contact with the liquids, such as, for example, the material out of which the chambers are manufactured, and, where applicable, the material used for the intermediate piece or the bottom piece, are hydrophilic, and the active substance carrier is made of a material which is hydrophobic. The previously presented surface energies apply accordingly.

Through this design of the hydrophobicity and/or the hydrophily of the materials, preferably a capillary effect is achieved, which leads to a good moistening of the active substance carrier and to a targeted and relatively complete transfer of the liquids from the chambers into the active substance carrier.

The fibers from which the above-mentioned fabrics, preferably non-wovens, are made, are not subject to any particular limitation and can be present, inter alia, as microfibers and/or hydrofibers.

Before opening the device, the active substance carrier can include a further active substance, which preferably has a high storage capability. The further active substance can, for example, be an antibiotic (e.g. fusidic acid, sulfonamides, gentamycin), honey, antiseptics (e.g. chlorhexidine, polyhexanide), analgesics (e.g. ibuprofen, lidocaine), collagen or hyaluronic acid. In a particular embodiment, the active substance carrier is free of a further active substance. Moreover, before the device is opened, the active substance carrier is dry so that a high liquid absorption take takes place. In a further embodiment, the active substance carrier is sterile before opening the device.

The dimensions of the active substance carrier can be adapted to the requirements, so that the thickness of the active substance carrier, for example, lies in the range of 0.5 to 20 mm, preferably in the range of 1 to 10 mm, the length lies in the range of 0.5 to 20 mm, preferably in the range of 1 to 10 cm, and the breadth lies in the range of 0.5 to 20 mm, preferably in the range of 1 to 10 cm. The surface area lies in the range of preferably 1 to 100 cm². The device can still be so equipped that larger or smaller wound dressings can be obtained. However, here, generally, the ease of handling the device is impaired. Moreover, very large wound dressings are relatively seldom requirements for patients who self-medicate, so that the manufacture of such devices is generally uneconomical.

The volume of liquid contained in the chambers can be adapted to the individual requirements and the size of the active substance carrier.

The active substance carrier is preferably moistened by the liquids. Here, the volume of liquid is preferably kept in an range, which does not lead to a release of drops from the skin or wound dressing comprising an active substance. Preferably, it may be provided that the separate chambers contain a total volume of liquid which leads to a oistening of the active substance carrier in the range of 0.05 ml/cm² to 0.8 ml/cm², particularly preferably in the range of 0.1 ml/cm² to 0.4 ml/cm² and very particularly preferably in a range of 0.15 ml/cm² to 0.3 ml/cm².

The not less than two liquids are contained in the minimum of two chambers, and the liquids are mixed when the device is opened and transferred to the active substance carrier. By mixing the minimum of two liquids, a mixture is obtained which contains the active substance. The active substance as such, or rather the liquid mixture, has a shorter shelf-life in comparison with that of the original liquids. Therefore, the present device is appropriate for all liquid active substances, which have a shorter shelf life, in comparison with that of a storage as two separate liquids.

Preferably, it may be provided that one of the liquids stored in the chambers, preferably the two liquids stored in the chambers, include water.

Some active substances are relatively unstable at the pH value at which the active substance is used, however at a lower or higher pH value they have good storage stability. In a preferred embodiment, it may be provided that the two liquids in the chamber exhibit different pH values, whereby the first liquid preferably has a pH value of at least 9, more preferably at least 10 and especially preferably at least 11, and the second liquid preferably has a pH value of a maximum of 4, more preferably a maximum of 3 and specially preferably a maximum of 2.

Examples of such active substances are described in the documents WO 00/48940 A1, WO 2005/049483 A2 and EP 1687238 B1, whereby these documents, in particular the wound treatment agents described therein, for disclosure purposes, are inserted by reference to these publications.

The dosage of the active substance can be adapted to the requirements of the patient, whereby this can be carried out preferably via the volume of liquid which is transferred to the active substance carrier, the concentration of active substance in the liquid and/or the size of the active substance carrier.

During a wound treatment, it may be provided that the dosage of active substance in the wound or skin dressing obtained is preferably chosen so that a change of the skin or wound dressing takes place within a time period of 6 to 48 hours, especially preferably within a time period of 12 to 36 hours and very especially preferably within a time period of 18 to 30. This can prevent destruction of the fibrils formed during the healing process without reducing the healing process or increasing other complications due to the wound fluid formed during healing process.

In a preferred embodiment, the components of the present device are essentially made up of medically harmless and cost-efficient plastics, whereby the individual components can be manufactured with corresponding methods. So, for example the components of the device previously and subsequently described here—a casing, a lid, an intermediate piece and/or a bottom piece—can be injection molded. Furthermore, the two chambers for storing two liquids are formed as a flat structure, preferably one or more films, which can be obtained by blow molding or extrusion.

The present device makes available a skin or wound dressing containing an active substance, whereby this can be present in all forms. The dressing can be made available, for example, as a compress or as a gauze.

Furthermore, the skin or wound dressing can be used alone or in combination with further components which are used for skin or wound treatment. Among these are, for example, alginates, hydrogels, hydrocolloids. Moreover, the skin or wound dressing can be used in combination with a further carrier component, for example with a foam component. Here, further substances, which promote the regeneration process or serve the well-being of the patient, can be made available. These components can include, for example, antibiotic (e.g. fusidic acid, sulfonamides, gentamycin), honey, antiseptics (e.g. chlorhexidine, polyhexanide), analgesics (e.g. ibuprofen, lidocaine), collagen or hyaluronic acid.

A further embodiment of the present invention is a wound treatment set containing a device, according to the invention, for the provision of a skin or wound dressing, a wound spacer, a cover film and a bandage fixation.

Wound spacers are known from the state of the art and prevent a wound dressing sticking to the wound, as this reduces wound healing when the dressing is changed. Wound spacers can, for example, be made as a silcone net, which, among others, can be obtained from Mölnlycke Health Care (product “Mepilex”).

Cover films (e.g., product “Opsite” from Smith & Nephew) prevent the active substance from leaking out upwards from the moist skin or wound dressing and thus not being available for wound healing. Accordingly, such films are generally waterproof, in case the active substance is an aqueous solution. Moreover these cover films are generally flexible so that they can be adapted to the specific requirements.

The dressing fixation serves to attach and safeguard the previously described components of the wound treatment set. For example, this can be carried out using the normal bandages, in particular roll bandages, linen bandages or gauze bandages, or bandage sleeves.

Another subject of the present invention is a method for providing a skin or wound dressing, including an active substance, in which a device according to the invention is opened, and a wound dressing containing an active substance is removed.

In a preferred embodiment of the present invention, the method can be carried out by operating one or two control elements, as has been described previously.

Hereinafter, using 9 figures, preferred embodiments of the present invention are described by way of example, without thereby limiting the invention.

Shown are:

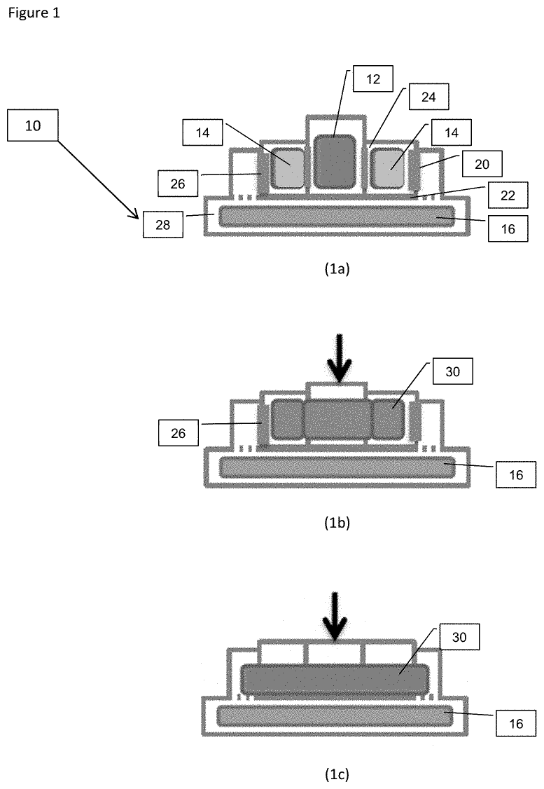

FIG. 1 a schematic longitudinal section representation of a first embodiment of a device according to the invention for providing a skin or wound dressing,

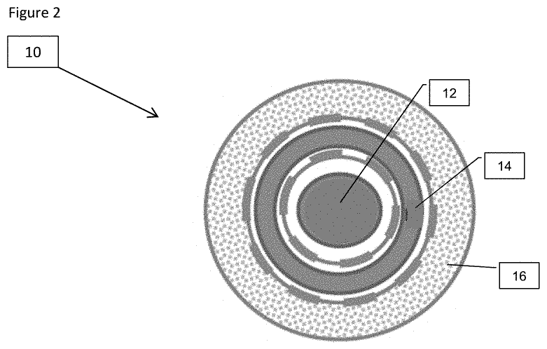

FIG. 2 a schematic cross-sectional representation of a first embodiment of a device according to the invention for providing a skin or wound dressing,

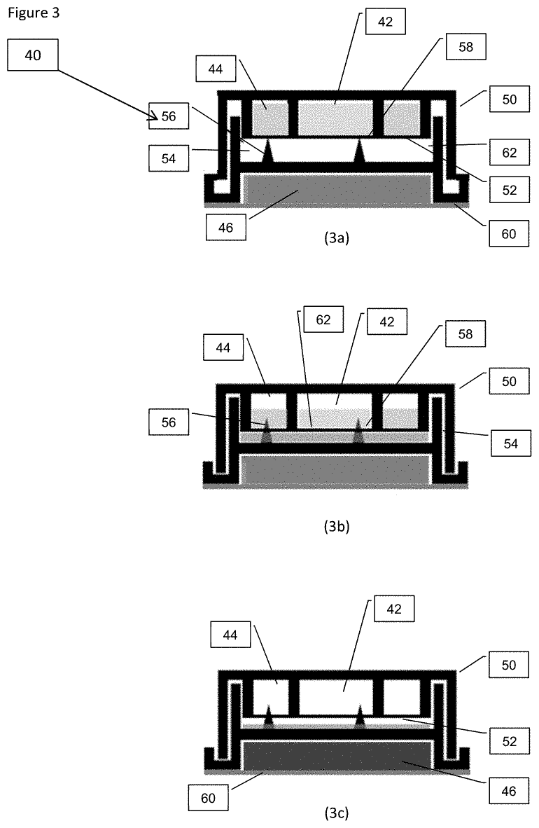

FIG. 3 a schematic longitudinal section representation of a second embodiment of a device according to the invention for providing a skin or wound dressing,

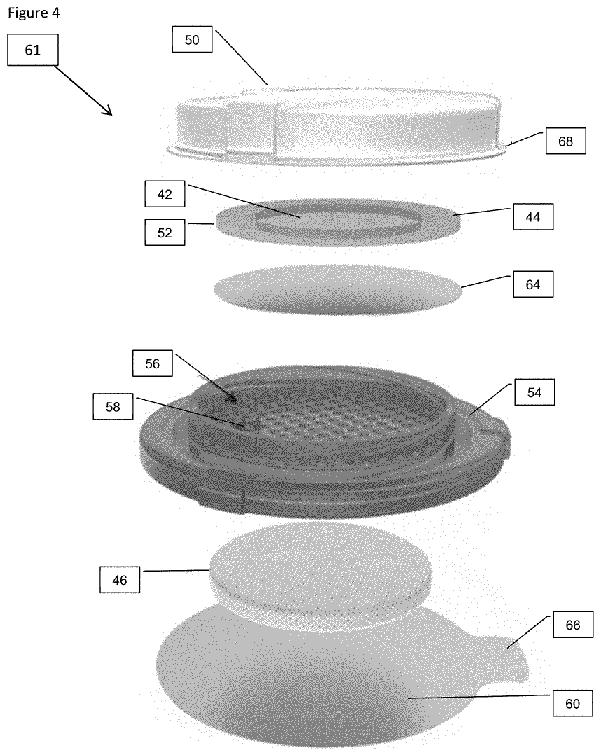

FIG. 4 a schematic exploded-view drawing of a third embodiment of a device according to the invention for providing a skin or wound dressing,

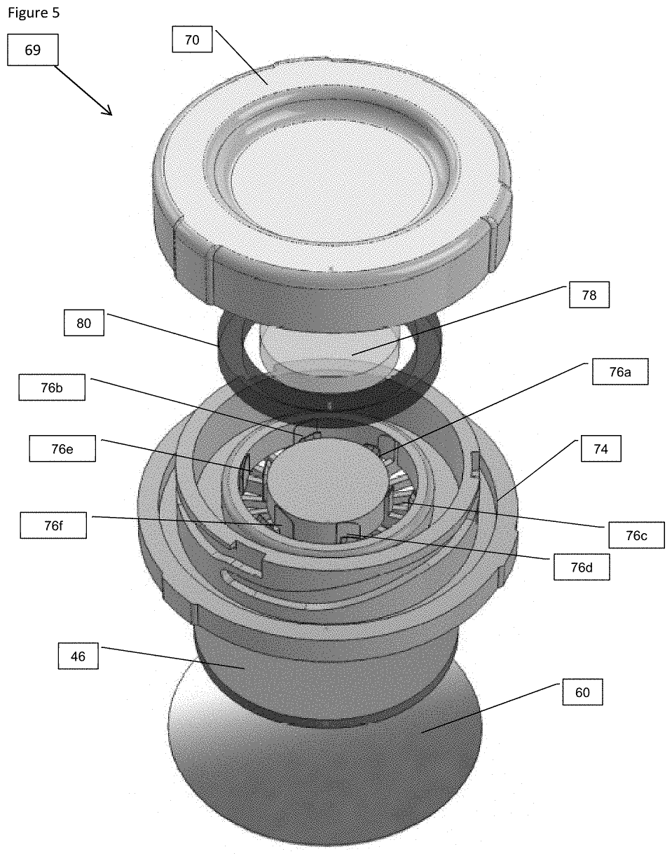

FIG. 5 a schematic exploded-view drawing of a fourth embodiment of a device according to the invention for providing a skin or wound dressing,

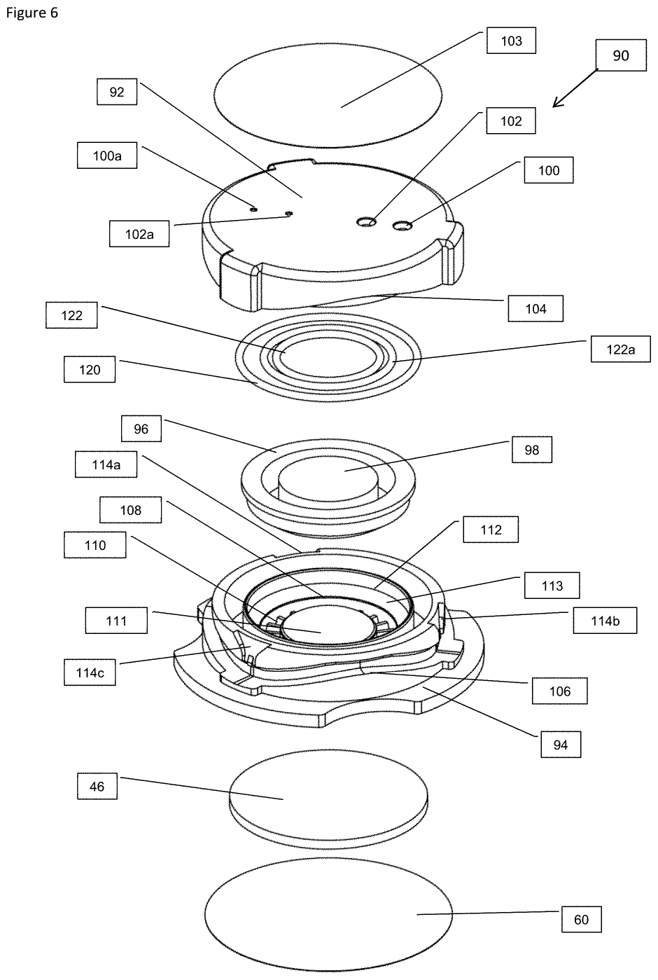

FIG. 6 a schematic exploded-view drawing of a fifth embodiment of a device according to the invention for providing a skin or wound dressing,

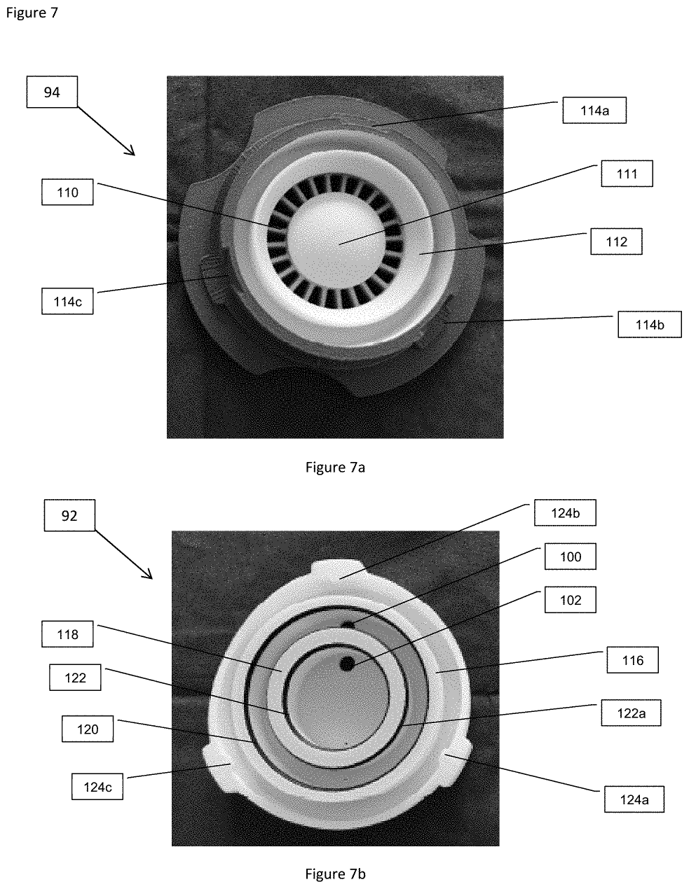

FIG. 7 representations of the intermediate piece and of the lid of the fifth embodiment of a device according to the invention for providing a skin or wound dressing,

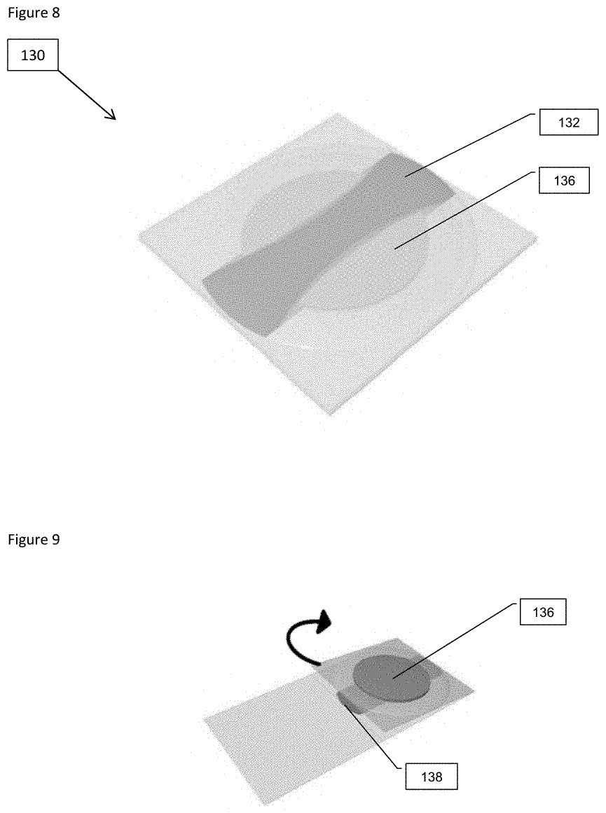

FIG. 8 a schematic top view representation of a sixth embodiment of a device according to the invention for providing a skin or wound dressing, in storable condition,

FIG. 9 a schematic top view representation of a sixth embodiment of a device according to the invention for providing a skin or wound dressing, in the condition where the two liquids are mixed,

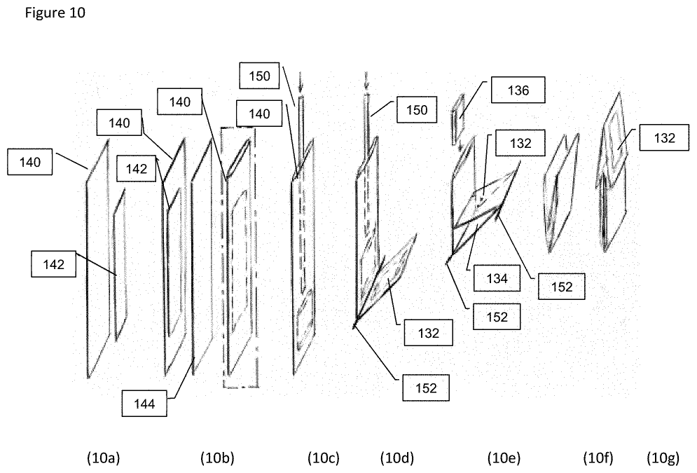

FIG. 10 a schematic representation of the manufacturing steps of a sixth embodiment of a device according to the invention for providing a skin or wound dressing,

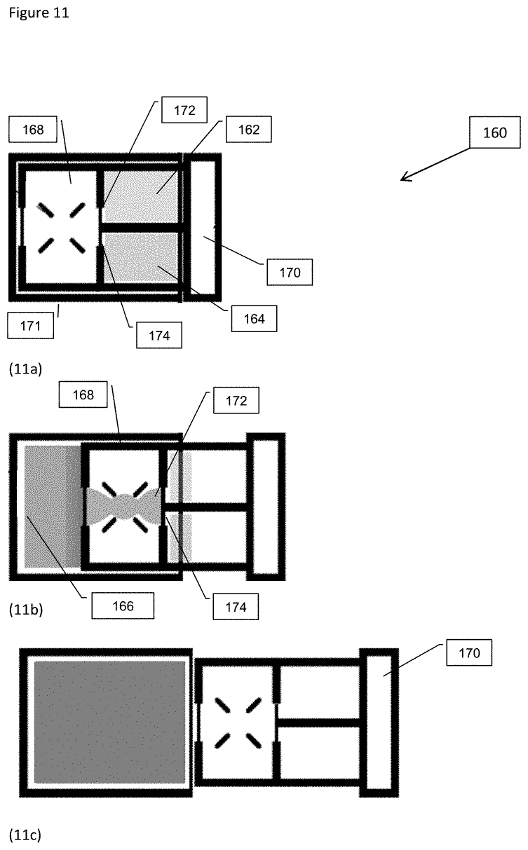

FIG. 11 a schematic cross-section representation of a seventh embodiment of a device according to the invention for providing a skin or wound dressing

FIG. 1 shows a schematic longitudinal section representation of a first embodiment of a device according to the invention for providing a skin or wound dressing, whereby FIG. 1a describes the storable condition of the device, FIG. 1b shows the condition where the two liquids are mixed, and FIG. 1c shows the condition of the device where a skin or wound dressing is presented which can be removed from the device.

The device for making available a skin or wound dressing 10, which is shown in FIG. 1a, includes at least two chambers for storing two liquids 12, 14, and an active substance carrier 16. The two chambers 12, 14 are formed in a lid 20, which together with an intermediate piece 22, encloses the two liquids. The intermediate piece 22 and the lid 20 have at least two predetermined breaking points 24, 26, whereby the first breaking point 24 is situated between the two chambers 12, 14 formed by the lid 20 and the intermediate piece 22. The second breaking point 26 is located between one of the chambers 12, 14 and the space 28 which contains the active substance carrier 16.

By applying pressure on the lid in the location of the first chamber 12, the first predetermined breaking point 24 will be specifically targeted and opened, so that the two chambers 12, 14 are connected to a mixing area 30. This half-opened condition is shown in FIG. 1b, whereby in this condition, the active substance carrier 16 has not yet been moistened. Here, the predetermined breaking point 26 is closed, so that at the beginning of pressure application this predetermined breaking point, first of all, resists opening.

If pressure is further applied, the second predetermined breaking point 26 will open, so that the condition shown in FIG. 1c is reached. Here, the liquid mixture which is contained in the mixing area 30 is transferred to the active substance carrier 16.

FIG. 2 shows a schematic cross-sectional view of a first embodiment, shown in FIG. 1, of a device according to the invention. It can be seen that the two chambers 12, 14 have a smaller diameter than the active substance carrier 16, so that, when the device is opened, at least some of the active substance is transferred to the active substance carrier 16 from above.

It should be noted that the first embodiment, shown in FIGS. 1 and 2, is presented relatively conceptually, in order to clarify the principles of the present invention. It goes without saying that the expert can implement this concept quite easily, whereby the chambers 12, 14 are executed in the form of a film arrangement, whereby the two predetermined breaking points 24, 26 are executed as detachable seams of different stability. Furthermore, pressure can be applied via a screw-in lid, as is explained in detail in the second and third embodiments.

FIG. 3 shows a schematic longitudinal section representation of a second embodiment of a device according to the invention for providing a skin or wound dressing, whereby FIG. 3a presents the storable condition of the device, FIG. 3b present the condition when the two liquids are mixed, and FIG. 3c presents the condition where a skin or wound dressing is made available which can then be removed from the device.

The device for providing a skin or wound dressing 40, which is shown in FIG. 3a, includes at least two chambers for the storage of two liquids 42, 44 and an active substance carrier 46. The two chambers 42, 44 are fitted in a lid 50 which, together with a separating film 52, encloses the two liquids. Below the separating film is an intermediate piece 54 with two protrusions 56, 58. The area between the separating film 52 and the intermediate piece 54 can be considered as the mixing area 62. Below the intermediate piece 54, in collaboration with a detachable bottom piece 60, a space is formed in which the active substance carrier 46 is introduced. The bottom piece 60, together with the intermediate piece 54, forms a casing.

In FIGS. 3b and 3c, the opening of the device shown in FIG. 3a is explained. As the lid 50 is screwed, in the direction of the intermediate piece 54, via a thread (not shown), the two protrusions 56, 58 are pressed through the separating film 52, so that the two chambers 42, 44 are opened and the two liquids flow into the mixing area 62.

The liquids stored in the chambers 42, 44 are conducted out of the mixing area 62 into the active substance carrier 46, so that a skin or wound dressing, including an active substance, is obtained. The gas previously contained in the active substance carrier and in the mixing area is conducted into the space between the lid 50 and the separating film 52, so that the liquids originally stored in the chambers 42, 44 are relatively completely pressed into the active substance carrier 46.

After the liquids stored in the chambers 42, 44 have been transferred relatively completely into the active substance carrier 46, the bottom piece 60 can be removed and the skin or wound dressing (with an active substance carrier 46, moistened with active substance) can be taken out of the device.

FIG. 4 shows a schematic exploded-view drawing of a third embodiment of a device according to the invention 61. The third embodiment corresponds conceptually with the embodiment form shown in FIG. 3, so that reference is made to this in the details, and the same reference numbers denote the same components. Thus the third embodiment comprises two chambers 42, 44 formed by the lid 50 and the separating film 52, an intermediate piece 54 with a thread, through which the lid 50 can be moved in the direction of the protrusions 56, 58 provided on the intermediate piece 54, in order to open the chambers 42, 44 and to release the two liquids. In the present exploded-view drawing, the formation of the two chambers 42, 44 is shown by corresponding sections on the separating film 52. In the third embodiment, the protrusions 56, 58 are in close proximity, so that the mixing of the liquids already takes place as the liquids flow out of the chambers. Furthermore, the third embodiment includes a distribution film 64, located between the intermediate piece 54 and the separation film 52 and which leads to an improved mixing of the two liquids. The active substance carrier 46, located below the intermediate piece 54, is held by a bottom piece 60 which is attachable to the intermediate piece 54 by gluing or welding, for example. The bottom piece 60, together with the intermediate piece 54, forms a casing. The bottom piece 60 has a grip portion 66, which is not covered by the active substance carrier. After opening the device, the bottom piece 60 can be detached from the intermediate piece 54, so that the skin or wound dressing, containing active substance can be easily taken out.

Moreover, the lid 50 is connected via an easily removable securing mechanism 68 with the intermediate piece 54, in order to prevent the device being opened unintentionally.

FIG. 5 shows a schematic exploded-view drawing of a fourth embodiment of a device according to the invention. The fourth embodiment corresponds conceptually with the embodiment form shown in FIG. 3, so that reference is made to this in the details, and the same reference numbers denote the same components. Thus the third embodiment comprises two chambers formed by the lid 70 and the separating film, not shown in FIG. 5, an intermediate piece 74 with a thread, through which the lid 70 can be moved in the direction of the protrusions 76 a, 76b, 76 c, 76 d, 76 e, 76 f provided on the intermediate piece 74, in order to open the chambers and to release the two liquids 78, 80. In the fourth the embodiment, there are provided a large number of protrusions 76 a, 76b, 76 c, 76 d, 76 e, 76 f, so that the film to be opened, after opening, has a large number of cuts. This results in a very complete transfer of the two liquids into the intermediate piece 74.

In comparison with the third embodiment, shown previously in FIG. 4, the intermediate piece 74 differs in that it exhibits a radially arranged sieve-like area, with an impervious inner zone. In the fourth embodiment, the protrusions 76 a, 76b, 76 c, 76 d, 76 e, 76 f are located on the radially arranged sieve-like areas, so that the mixing of the two liquids takes place in this radially arranged sieve-like area. This embodiment is particularly useful for active substances where a pH change takes place during mixing. Preferably, this embodiment can be used for active substances which have a long shelf life at a high or at a low pH value. A skin tolerable pH value is appropriate and necessary however for the application of the active substances, whereby this can be achieved by mixing two liquids for pH adjustment.

Below the intermediate piece 74, the active substance carrier 46 is located and it is held by a bottom piece 60 which is attachable to the intermediate piece 74 by gluing or welding, for example. The bottom piece 60, together with the intermediate piece 74, forms a casing. As previously shown, the bottom piece 60 can have a grip portion, which is not covered by the active substance carrier. After opening the device, the bottom piece 60 can be detached from the intermediate piece 74, so that the skin or wound dressing containing active substance can be easily taken out.

Moreover, it can be provided that the lid 70 is connected via an easily removable securing mechanism 68 with the intermediate piece 74, in order to prevent the device being opened unintentionally.

FIG. 6 shows a schematic exploded-view drawing of a fifth embodiment of a device according to the invention. The fifth embodiment corresponds conceptually with the embodiment form shown in FIG. 3, so that reference is made to this in the details, and the same reference numbers denote the same components.

The device for providing a skin or wound dressing 90, which is shown in FIG. 6, comprises at least two chambers for the storage of two liquids and an active substance carrier 46. The two chambers are formed in a lid 92 which, together with the intermediate piece 94, contains the two liquids 96, 98. In the present embodiment ring shaped walls (not shown in FIG. 6) are provided in lid 92 which are fitted with seals 120, 122 and 122a. The seals are 120, 122 and 122a are located between the intermediate piece 94 and the ring-formed walls (not shown in FIG. 6). Instead of a separate seal, a sealed area can also be formed using appropriate material selection and/or shaping. Here, two chambers are created which can be filled via openings 100 and 102 in the lid. Two further openings 100a and 102a, through which air can escape, simplify the filling of the chambers. After filling, in the present embodiment, the openings 100, 100a, 102 and 102a are sealed with a film 103. The lid is movable upwards by a turning movement, so that the ring form walls, fitted with a seal, located in lid 92, can be moved away from intermediate piece 94 creating an opening. Here, a suitable thread is provided in the intermediate piece 94 and in the lid 92, which is formed by helical or wavy shapes 104 and 106, which convert a turning motion into a linear motion. The area between lid 92 and intermediate piece 94 can be considered as mixing area 108. Intermediate piece 94 has a radially aligned sieve-like area 110, whereby an impervious inner zone 111 exists, which, together with the lid forms a first chamber. The second chamber is formed via an annular wall 112 together with the lid. The annular wall 112 has a radial circumferential bevel 113, so that, after opening, the liquid in the second chamber flows easily into the radially aligned sieve-like area 110. Below intermediate piece 94, in cooperation with a removable bottom piece 60, a space is formed in which the active substance carrier is introduced. The bottom piece 60, together with the intermediate piece 94, forms a casing.

Furthermore, intermediate piece 94 has three recesses 114a, 114b, 114c, which prevent an unintentional opening of the device. The counterparts for tongues, not shown in FIG. 6, are located in lid 92.

FIG. 7 shows a representation of the intermediate piece (FIG. 7a) and of the lid (FIG. 7b) of the fifth embodiment in a schematic exploded-view.

In FIG. 7a, a top view of intermediate piece 94 is shown. The intermediate piece 94 exhibits a radially arranged sieve-like area 110 with an impervious inner zone 111, which, together with the lid, forms the first chamber. The second chamber is formed via an annular wall 112 together with the lid. The annular wall 112 has a radial circumferential bevel, so that, after opening, the liquid in the second chamber flows easily into the radially aligned sieve-like area 110.

Furthermore, three recesses 114a, 114b, 114c, are shown in FIG. 7a which prevent an unintentional opening of the device.

FIG. 7b shows the inner area of 92. In particular the annular walls 116, 118 are shown which together with the seals 120, 122, 122a, form two chambers with the previously presented sections of the intermediate piece. The chambers can be filled via the openings 100 and 102 in the lid.

Moreover, the lid exhibits three tongues 124a, 124b, 124c, which, together with the previously described recesses in the intermediate piece, prevent an unintentional opening of the device.

FIG. 8 shows a schematic top view representation of a sixth embodiment of the device according to the invention 130. In this sixth embodiment of the device in storage condition, the active substance carrier 136 is located between the two chambers for the storage of the two liquids, where, on the basis of the representation available, merely chamber 132 is shown. The chambers are formed by various polymer films, which are made into cavities. The sixth embodiment is fitted with an elastic element, through which, part of the storable device for providing a skin or wound dressing is under tension. The tension can be described with a molecular memory effect, through which the plastic returns to its original shape. Here, by releasing a safety mechanism, the opening of the chambers for storing two liquids is facilitated, preferably effected.

In FIG. 9, for further clarification, the condition of the sixth embodiment is shown as the two liquids are mixed. By releasing a safety mechanism, the chambers where the liquids are stored are first of all connected. This creates mixing space 138, which by further opening is connected with the cavity where active substance carrier 136 is located. The unfolding/opening for connecting the two chambers filled with liquid and/or the unfolding/opening to connect mixing space 138 with the cavity containing active substance carrier 136, are supported by the elastic element, preferably effected by it. In this preferred embodiment, by disengagement of a weld or another securing element, due to the tension, an opening process is caused with which the chambers are each connected with each other.

FIG. 10 shows a schematic representation of the manufacturing steps of a sixth embodiment of a device according to the invention for providing a skin or wound dressing.

FIG. 10a shows that, first of all, a flexible polymer film 140 is joined with an elastomeric film 142. This can be done preferably by two-dimensional gluing. The elastomeric film 142 has a smaller surface area than the flexible polymer film 140. Subsequently, as shown in FIG. 10b, the flexible polymer film 140 is joined to the stiff polymer film 144 at three edges. This can be carried out, for example, by heated laser welding. In this way, an elongated cavity is created which is closed on three sides. Subsequently, using a pipette 150, first of all the first liquid is filled into this cavity, as shown in in FIG. 10c. After this, the filled area is bent off and separated with a clamp 152, so that the first chamber 132 is created which contains the first liquid. In the following, as shown in FIG. 10d, the second liquid is filled into the remaining area of the cavity.

After the cavity has been filled, the area filled with second liquid is also bent off and closed with a clamp 152, so that a second chamber 134 is created. Subsequently, the active substance carrier 136 is placed in the remaining space of the cavity, as shown in FIG. 10e.

Subsequently, the cavity is closed and a strap is attached with which this closure can be easily opened, as shown in FIG. 10f. According to FIG. 10g, the chambers which have been created can be turned over in such a way that the active substance carrier 136 is located between chambers 132, 134 with the liquids. After this, a seal can be fitted which prevents an unintentional opening of the device. After the seal has been fitted, the clamps generally can be removed.

The sixth embodiment comprises a flexible polymer film 140, an elastic element in the form of an elastomeric film 142 and a stiff polymer film 144. These polymer films 140, 142 and 144 are connected in such a way that two chambers 132, 134 are created, in which the two liquids are stored, as well as a cavity into which the active substance carrier 136 is placed.

In a preferred embodiment, the chambers are merely separated by clamping pressure, which, after the securing element has been released, leads first to an opening of the chambers forming a mixing area, and subsequently to an opening of the connection between the mixing area and the cavity.

FIG. 11 shows a schematic cross-section representation of a seventh embodiment of a device according to the invention for providing a skin or wound dressing 160, whereby FIG. 11a shows the storable condition of device 160; FIG. 11b shows the condition when the two liquids are mixed, and FIG. 11c shows the condition of the device, in which a skin or wound dressing containing an active substance is provided, which can be removed from the device.

The device shown in FIG. 11a for providing a skin or wound dressing includes at least two chambers for the storing the two liquids 162, 164 and a mixing chamber 168, whereby the active substance carrier is located below the chambers 162, 164 and the mixing chamber 168. The two chambers 162, 164 are connected to a pulling mechanism 170, so that a pulling movement leads to the opening of two predetermined breaking points 172, 174. The pulling mechanism can be safeguarded, for example by an easily releasable predetermined breaking point, whereby this can connect the pulling mechanism 170 with the casing 171.

After the predetermined breaking points 172, 174 have been opened the liquids flow into the mixing chamber 178 and from there to the active substance carrier 176, as shown in FIG. 11b.

After the pulling mechanism 170 has been pulled out completely, the active substance containing skin or wound dressing can be taken out, as shown in FIG. 11c.

The features of the invention disclosed in the previous description, as well as in the claims, Figures and execution examples, can, both individually or in any form of combination, be essential for the realization of the invention in its various embodiments.

1. A device for providing a skin or wound dressing, comprising an active substance, wherein the device comprises at least two chambers for the storage of at least two liquids, which are openable by mechanical action, so that, after opening of the chambers, the liquids are miscible and are transferrable to an active substance carrier.

2. A device according to claim 1, wherein after opening of the chambers, the liquids are mergeable outside the active substance carrier, or a mixing area is present into which the liquids are transferrable, and the liquids are then transferrable from the mixing area to the active substance carrier.

3. A device according to claim 1, wherein the at least two separate chambers are manufactured from a plastic.

4. A device according to claim 1, wherein the two chambers are openable by pressure, whereby the released liquids are transferrable to the active substance carrier.

5. A device according to claim 1, wherein the at least two separate chambers are openable by a turning movement or linear movement, whereby the chambers are mechanically opened by a protruding edge, a sealed area, or by a predetermined breaking point.

6. A device according to claim 1, wherein the two chambers and the active substance carrier are arranged in at least two different levels.

7. A device according to claim 1, wherein the device has a casing which encloses the active substance carrier.

8. A device according to claim 1, wherein the device comprises at least one lid, which with an intermediate piece, encloses the at least two separate chambers for the storage of two liquids; or these chambers are formed in the lid or the intermediate piece.

9. A device according to claim 8, wherein the active substance carrier is located below the intermediate piece, whereby the active substance carrier is held by an openable bottom piece.

10. A device according to claim 8, wherein the intermediate piece has several openings.

11. A device according to claim 1, wherein the device includes at least one elastic element, whereby the release of a seal initiates a movement whereby the opening of the chambers for storing two liquids is facilitated.

12. A device according to claim 11, wherein the two chambers are formed by at least two plastic layers, which are firmly connected on two opposite sides, and, on one side with which the chambers can be connected are separated by a folding edge.

13. A device according to claim 11, wherein the active substance carrier is contained in a third chamber, which is formed by at least two plastic layers, which are firmly connected on two opposite sides and on one side are separated by a folding edge, from at least one further chamber which can be designed as a mixing chamber.

14. Wound treatment set including a device according to claim 1, a wound spacer, a cover film, and a dressing fixation.

15. Method for providing a skin or wound dressing, including an active substance, comprising opening a device according to claim 1, and taking out a wound dressing dosed with an active substance.

16. A device according to claim 2, wherein the liquids are mergeable outside the active substance carrier by creating a mixing area upon opening of the two chambers.

17. A device according to claim 3, wherein the at least two separate chambers are manufactured from a plastic with one section made out of a flexible plastic.

18. A device according to claim 17, wherein the at least two separate chambers are manufactured from a plastic film.

19. A device according to claim 4, wherein the two chambers are openable by pressure, whereby the released liquids are transferrable to the active substance carrier with the aid of this pressure.

20. A device according to claim 5, wherein the at least two separate chambers are openable by a turning movement or linear movement, whereby the chambers are mechanically opened by an openable welding seam.

21. A device according to claim 9, wherein the active substance carrier is located below the intermediate piece, whereby the active substance carrier is held by an openable bottom piece, whereby the bottom piece is impervious to liquids.

22. A device according to claim 21, wherein the active substance carrier is located below the intermediate piece, whereby the active substance carrier is held by an openable bottom piece, whereby the bottom piece has a grip portion which is not covered by the active substance carrier.

23. A device according to claim 10, wherein the intermediate piece is of a sieve-like design.

24. A device according to claim 11, wherein the device includes at least one elastic element, whereby the release of a seal initiates a folding mechanism, whereby the opening of the chambers for storing two liquids is facilitated.

25. A device according to claim 11, wherein the device includes at least one elastic element, whereby the release of a seal initiates a movement, whereby the opening of the chambers for storing two liquids is effected.
